(19) United States
(12) Patent Application Publication
(10) Pub. No.: US 2026/0167892 A1
(43) Pub. Date: Jun. 18, 2026

(54) WATERLESS CLEANSING COMPOSITION

(71) Applicant: PAPA SOAP LLC, Groton, CT (US)

(72) Inventors: Ruslans Maidans, Stonington, CT (US); Alexey Rostovtsev, Lima (PE); Tim Drennan, Denver, CO (US)

(73) Assignee: PAPA SOAP LLC, Groton, CT (US)

(21) Appl. No.: 19/418,944

(22) Filed: Dec. 13, 2025

Related U.S. Application Data
(60) Provisional application No. 63/735,125, filed on Dec. 17, 2024.

(51) Int. Cl. C11D 1/10; C11D 3/20; C11D 3/37

(52) U.S. Cl. CPC C11D 1/10; C11D 3/2079; C11D 3/2093; C11D 3/2096; C11D 3/3707

(57) ABSTRACT

A rinse-free cleansing composition and method of use are disclosed. The composition comprises an aqueous carrier, a synergistic rheology system, an emollient, a mild surfactant, and a preservative system. The rheology system includes a cellulose derivative, a gum, and a film-forming polymer. Upon application to a surface, such as skin, and subsequent rubbing, the composition rapidly forms solid, peelable coagulants. These coagulants encapsulate and lift impurities, such as dirt and oils, and then detach from the surface without the need for rinsing with water or wiping with a towel, leaving the surface clean. The composition is formulated to be gentle, stable, and effective for waterless hygiene.

### BACKGROUND OF THE INVENTION

### Field of the Invention

The present invention relates generally to the field of cleansing compositions. More particularly, it pertains to rinse-free, waterless compositions formulated for cleaning skin and other surfaces, which do not require the use of water or towels for removal.

### Description of Related Art

Effective personal hygiene, particularly hand cleaning, is a cornerstone of public health, preventing the spread of pathogens and contaminants. The conventional and most widely accepted method for cleaning hands involves the use of soap and water. This method is highly effective at removing not only microorganisms but also physical dirt, grime, oils, and other debris. However, the primary drawback of this method is its absolute dependence on a readily available source of clean water and a means for drying, such as a towel. In many situations, such as during travel, outdoor recreational activities, disaster zones, or in regions with limited water infrastructure, access to such resources is not guaranteed, making conventional hand washing impractical or impossible. Furthermore, the significant water consumption associated with frequent hand washing contributes to environmental concerns, particularly in areas experiencing water scarcity.

To address the limitations of water-based cleaning, alcohol-based hand sanitizers were developed. These products offer the convenience of waterless application and are effective at killing a broad spectrum of germs. However, their mechanism of action is primarily antimicrobial; they are largely ineffective at removing physical soil, grease, or other particulate matter from the skin. Furthermore, alcohol-based sanitizers have significant drawbacks. They are flammable, posing a hazmat and shipping burden; their high alcohol content can cause skin irritation and dermal barrier damage with overuse; and they are ineffective against certain resilient or spore-forming pathogens, such as Clostridium difficile and norovirus. Overuse may also contribute to microbial resistance. The debris, dirt, and pathogens remain on the skin surface, which can be undesirable both aesthetically and hygienically.

The art has also seen the development of so-called “waterless” or “rinse-free” hand cleaners that aim to remove physical dirt without water. Some of these compositions are designed to be applied and then wiped off with a towel, which fails to provide a truly self-contained, stand-alone cleaning system and does not eliminate the need for an external cleansing aid. Other formulations, such as that described in U.S. Pat. No. 5,259,984 to Hull, disclose a “spread-on/rub-off” mechanism where the composition “beads up” and falls away from the skin. While this represents an advancement, such prior art compositions rely on ingredients that are largely synthetic and may invoke negative reactions. For example, the composition in Hull utilizes alkali metal hydroxides to achieve a high pH, which can cause contact dermatitis, and synthetic surfactants like alkylphenol ethoxylates, which have faced scrutiny for environmental and health reasons. The Hull patent also relies on methanol, which is well-known to increase the toxicity of cosmetics, and the reliance of Hull on isopropyl alcohol as a solvent may cause further sensitization to already damaged skin.

Additionally, such formulations have difficulty in controlling rheological properties for even skin distribution, as gel syneresis (separation of gel and liquid) commonly prevents complete encapsulation of dirts and oils. Moreover, the process described requires a rubbing time of up to five minutes for the product to bead up and fall off, a duration that may be impractical for convenient, on-the-go use. Other prior art compositions are designed to leave a persistent film in a “glove-forming” composition on the skin rather than shedding completely, which does not provide a truly clean feeling and is impractical in an active, outdoor setting.

Therefore, a significant need exists in the art for a cleansing composition that overcomes the deficiencies of the prior art. There is a need for a product that is truly waterless, requiring neither rinsing nor wiping, and is effective at removing both microbial contaminants and physical impurities like dirt and grease. Furthermore, there is a demand for such a product to be fast-acting, composed of gentle ingredients, and to provide a pleasant user experience with a smooth, clean after-feel.

### SUMMARY OF THE INVENTION

The present invention provides a novel waterless cleansing composition that addresses the aforementioned limitations of the prior art. The composition is a stable, aqueous-based formulation that effectively cleans surfaces such as skin without the need for subsequent rinsing with water or wiping with an external agent, such as a towel.

This composition is mechanistically distinct from conventional soaps, which require water as a carrier for the removal of surfactants and soils. The present invention circumvents the water carrier principle by providing a self-contained system that utilizes a drying-and-coagulation mechanism, where the composition itself forms the aggregates that carry away impurities.

In one aspect, the invention is a rinse-free cleansing composition comprising a primary aqueous carrier, a unique rheology system, an emollient, a mild surfactant system, and a preservative system. The composition is distinguished by its synergistic system of components, which work in concert to achieve a novel cleansing mechanism. The rheology system comprises a combination of polymers, such as a cellulose derivative, a gum, and a film-forming polymer or starch, which together create a specific matrix structure that retains its moisture until application. This matrix is modulated by an emollient, which contributes to a pleasant skin feel and controls adhesion, and a mild surfactant, which provides cleansing action without disrupting the matrix's structural integrity.

The key functional advantage of the composition is its ability to form solid coagulants or peelings upon being rubbed on a surface. This mechanical action causes the composition's matrix to coagulate around dirt, oils, and other impurities, effectively encapsulating them. As rubbing continues, these solid coagulants cleanly detach from the surface, falling away as small, dry particles and leaving the underlying surface clean, smooth, and refreshed. This entire process is rapid, typically completed in under two minutes.

In another aspect, the invention provides a method for cleaning a surface without rinsing. The method involves applying a quantity of the aforementioned composition to the surface, rubbing the composition for a short duration, such as approximately 30 seconds to 1.5 minutes, and allowing the solid particles that form to fall away from the surface. This method provides a convenient, effective, and environmentally conscious alternative to traditional washing methods. The composition's formulation, based on gentle ingredients, makes it particularly suitable for use on human skin.

### DETAILED DESCRIPTION OF THE INVENTION

The present invention is directed to a waterless cleansing composition, typically in the form of a gel, lotion, or cream, that can be applied to a surface to remove impurities without the need for rinsing. The following detailed description illustrates various embodiments of the invention. Unless otherwise noted, all percentage values described herein refer to percentages by weight (% wt) based on the total weight of the composition.

### I. Overview of the Composition

The cleansing composition of the present invention is an aqueous-based formulation comprising several key functional components that work synergistically to provide its unique cleansing action. The constituent parts can be broadly categorized as: a primary aqueous carrier, a rheology system (comprising one or more thickening agents), one or more functional ingredients (such as emollients and fragrances), a surfactant system, and a preservative system. The specific selection and concentration of these components allow the final product to be tailored for desired consistency, cleansing efficacy, and sensory characteristics.

### II. Component Details and Alternatives

The composition of the present invention is defined by the specific combination of its components, which are selected to be gentle and effective. Table 1 below summarizes the component categories, their functional ranges, an exemplary embodiment, and suitable alternatives for each component.

TABLE 1
Composition Ingredients and Weight Percentages

| | Broad | Preferred | Exemplary | |
|---|---|---|---|---|
| Component | Weight % | Weight % | Embodiment | Suitable |
| Category | Range | Range | (% wt) | Alternatives |
| Primary | 50%-90% | 60%-85% | Reverse | Deionized |
| Carrier | | | Osmosis (RO) | water, distilled |
| | | | Water | water, purified |
| | | | (79.30%) | water, floral |
| | | | | hydrosols (e.g., |
| | | | | rosewater, |
| | | | | lavender |
| | | | | water), and |
| | | | | specialty |
| | | | | waters |
| Rheology | 1%-12% | 4%-10% | | |
| System | | | | |
| Cellulose | 1%-8% | 3%-5% | Hydroxyethyl- | Carboxymethyl |
| Thickener | | | cellulose (HEC) | cellulose, |
| | | | (4.00%) | hydroxypropyl |
| | | | | cellulose, |
| | | | | methyl |
| | | | | cellulose, |
| | | | | pullulan, |
| | | | | hydroxypropyl |
| | | | | methylcellulose |
| | | | | (HPMC), and |
| | | | | sodium |
| | | | | carboxymethyl |
| | | | | cellulose |
| | | | | (SCMC) |
| Gum Thickener | 0.2%-3% | 0.5%-1.5% | Organic | Guar gum, |
| | | | Xanthan Gum | locust bean |
| | | | (1.00%) | gum, |
| | | | | carrageenan, |
| | | | | gellan gum, |
| | | | | tara gum, |
| | | | | konjac gum, |
| | | | | pullulan, |
| | | | | cellulose gum, |
| | | | | sclerotium |
| | | | | gum, and |
| | | | | tragacanth |
| Third Polymer | 2%-8% | 3%-5% | Tapioca | Cornstarch, |
| Component | | | (Arrowroot) | potato starch, |
| (Starch/Film- | | | Starch (4.00%) | rice starch, oat |
| Former) | | | | starch, |
| | | | | arrowroot |
| | | | | powder, |
| | | | | polyvinyl |
| | | | | alcohol, |
| | | | | polyacrylates |
| Functional | 1%-20% | 5%-13% | | |
| Ingredients | | | | |
| Emollient | 1%-15% | 5%-12% | Floramac 10 | Caprylic/capric |
| | | | (Ethyl | triglyceride, |
| | | | Macadamiate) | jojoba esters, |
| | | | (8.00%) | squalane, |
| | | | | sunflower oil |
| | | | | esters, almond |
| | | | | oil, apricot |
| | | | | kernel oil, |
| | | | | avocado oil, |
| | | | | borage seed |
| | | | | oil, canola oil, |
| | | | | castor oil, |
| | | | | coconut oil, |
| | | | | corn oil, |
| | | | | evening |
| | | | | primrose oil, |
| | | | | grape seed oil, |
| | | | | hazelnut oil, |
| | | | | hemp seed oil, |
| | | | | jojoba oil, |
| | | | | kukui nut oil, |
| | | | | lanolin, |
| | | | | macadamia |
| | | | | nut oil, mineral |
| | | | | oil, olive oil, |
| | | | | palm oil, |
| | | | | safflower seed |
| | | | | oil, sesame oil, |
| | | | | shea butter, |
| | | | | soybean oil, |
| | | | | cetyl alcohol, |
| | | | | stearyl alcohol, |
| | | | | isopropyl |
| | | | | myristate, and |
| | | | | isopropyl |
| | | | | palmitate |
| Fragrance | 0.1%-0.5% | 0.15%-0.25% | Linalool | Essential oils |
| | | | (0.20%) | (e.g., lavender, |
| | | | | citrus), |
| | | | | geraniol, |
| | | | | fragrance-free |
| | | | | formulations |
| Surfactant | 0.5%-6% | 1%-4% | | |
| System | | | | |
| Mild Surfactant | 0.5%-6% | 1%-4% | Sodium Cocoyl | Sodium lauroyl |
| | | | Glutamate | sarcosinate, |
| | | | (2.00%) | disodium |
| | | | | cocoyl |
| | | | | glutamate, |
| | | | | coco glucoside, |
| | | | | decyl |
| | | | | glucoside, |
| | | | | ammonium |
| | | | | laureth sulfate, |
| | | | | ammonium |
| | | | | lauryl sulfate, |
| | | | | cocamidopropyl |
| | | | | betaine, |
| | | | | disodium |
| | | | | cocoamphodiacetate, |
| | | | | sodium |
| | | | | C14-16 olefin |
| | | | | sulfonate, |
| | | | | sodium |
| | | | | cocoamphoacetate, |
| | | | | sodium cocoyl |
| | | | | isethionate, |
| | | | | sodium laureth |
| | | | | sulfate, sodium |
| | | | | lauroamphoacetate, |
| | | | | sodium lauroyl |
| | | | | lactylate, |
| | | | | sodium lauryl |
| | | | | sulfate, and |
| | | | | sodium methyl |
| | | | | cocoyl taurate |
| Preservative | 0.1%-5% | 0.3%-2.5% | | |
| System | | | | |
| Preservative | 0.1%-5% | 0.3%-2.5% | Geogard Ultra | Phenoxyethanol, |
| | | | (1.50%) | potassium |
| | | | | sorbate, |
| | | | | sodium |
| | | | | benzoate (as a |
| | | | | single agent), |
| | | | | ethylhexylglycerin, |
| | | | | benzoic acid, |
| | | | | benzyl alcohol, |
| | | | | bronopol, |
| | | | | butylparaben, |
| | | | | chlorphenesin, |
| | | | | dehydroacetic |
| | | | | acid, |
| | | | | diazolidinyl |
| | | | | urea, DMDM |
| | | | | hydantoin, |
| | | | | imidazolidinyl |
| | | | | urea, |
| | | | | iodopropynyl |
| | | | | butylcarbamate, |
| | | | | methylchlorois |
| | | | | othiazolinone, |
| | | | | methylisothiazolinone, |
| | | | | methylparaben, |
| | | | | and |
| | | | | propylparaben |

### A. Primary Carrier

The primary carrier serves as the solvent base for the composition and typically constitutes the largest portion by weight. In a preferred embodiment, the carrier is high-purity water, such as reverse osmosis (RO) water, deionized water, or distilled water, present in an amount from about 50% to about 90% by weight, and more preferably from about 60% to about 85% by weight. In certain embodiments, a portion of the water, for example up to 10%, may be substituted with specialty waters like floral hydrosols to impart additional skin-conditioning benefits or a natural fragrance.

### B. Rheology System

A key aspect of the invention is the synergistic rheology system, which provides the composition's gel-like structure and is critical to the formation of the peelable coagulants. This novel system is a combination of at least three types of thickening agents: a cellulose derivative, a gum, and a third polymer component (such as a starch or synthetic film-former), which work in concert for a rapid reaction when the product is applied.

The cellulose derivative, such as hydroxyethylcellulose (HEC), provides the primary viscosity and body to the composition. It is typically present in a range of about 1% to about 8% by weight. Suitable alternatives include other cellulose ethers like carboxymethylcellulose, hydroxypropyl cellulose, methyl cellulose, pullulan, hydroxypropyl methylcellulose (HPMC), and sodium carboxymethyl cellulose (SCMC).

The gum, such as xanthan gum, acts as a stabilizer, preventing syneresis (weeping) and improving the texture and cohesiveness of the gel matrix. It is typically used in a concentration of about 0.2% to about 3% by weight. Other suitable gums include guar gum, locust bean gum, carrageenan, gellan gum, tara gum, konjac gum, pullulan, cellulose gum, sclerotium gum, and tragacanth.

The third polymer component, which may be a starch, such as tapioca (arrowroot) starch, is a critical element of the formulation. It enhances the texture, provides a smooth, matte feel upon application, and aids in the coagulation and drying process that forms the solid peelings. The starch is typically present in an amount of about 2% to about 8% by weight. Alternative starches include cornstarch, potato starch, rice starch, oat starch, and arrowroot powder, the latter of which may also provide skin-soothing benefits. In other embodiments, this component may be a synthetic film-forming polymer, such as polyvinyl alcohol or a polyacrylate, which also aids in the coagulation and formation of solid peelings.

### C. Functional Ingredients

Functional ingredients are included to improve the user experience and performance of the composition.

An emollient is incorporated to provide a smooth, silky skin feel during and after application, counteracting any potential drying effect and ensuring the coagulants do not adhere too strongly to the skin. In a preferred embodiment, the emollient is Ethyl Macadamiate, sold under the trade name Floramac 10. This plant-derived ester provides a light, non-greasy feel comparable to silicones and is present in a range of about 1% to about 15% by weight. Alternatives include other light esters or lipids such as caprylic/capric triglyceride, jojoba esters, squalane, sunflower oil esters, almond oil, apricot kernel oil, avocado oil, borage seed oil, canola oil, castor oil, coconut oil, corn oil, evening primrose oil, grape seed oil, hazelnut oil, hemp seed oil, jojoba oil, kukui nut oil, lanolin, macadamia nut oil, mineral oil, olive oil, palm oil, safflower seed oil, sesame oil, shea butter, soybean oil, cetyl alcohol, stearyl alcohol, isopropyl myristate, and isopropyl palmitate.

A fragrance component may be added for aesthetic appeal. In one embodiment, this is linalool, a naturally occurring terpene alcohol with a pleasant floral scent, used at a concentration of about 0.1% to about 0.5% by weight. Other essential oils or fragrance components can be used, or the composition can be formulated to be fragrance-free for sensitive users.

### D. Surfactant System

The composition includes a mild surfactant to aid in lifting and encapsulating dirt, oil, and other impurities from the skin. The surfactant must be effective yet gentle enough not to cause irritation or disrupt the skin's natural barrier. In a preferred embodiment, the surfactant is sodium cocoyl glutamate, an amino acid-based surfactant known for its mildness and excellent cleansing properties. It is typically present in a range of about 0.5% to about 6% by weight. Suitable alternatives include other mild surfactants such as sodium lauroyl sarcosinate, disodium cocoyl glutamate, coco glucoside, decyl glucoside, ammonium laureth sulfate, ammonium lauryl sulfate, cocamidopropyl betaine, disodium cocoamphodiacetate, sodium C14-16 olefin sulfonate, sodium cocoamphoacetate, sodium cocoyl isethionate, sodium laureth sulfate, sodium lauroamphoacetate, sodium lauroyl lactylate, sodium lauryl sulfate, and sodium methyl cocoyl taurate.

### E. Preservative System

To ensure microbiological stability and provide an adequate shelf life, a broad-spectrum preservative system is essential. The selection of a preservative that is effective and gentle is an important feature. A preferred embodiment utilizes Geogard Ultra, a patented blend of gluconolactone and sodium benzoate. This system is ECOCERT/COSMOS-accepted and provides broad-spectrum protection. The gluconolactone component also offers moisturizing benefits. This specific preservative system is another point of novelty for the composition. It is typically used in a concentration of about 0.3% to about 2.5% by weight. Alternative preservative systems acceptable under relevant regulatory standards may also be used, such as phenoxyethanol, potassium sorbate, sodium benzoate (as a single agent), ethylhexylglycerin, benzoic acid, benzyl alcohol, bronopol, butylparaben, chlorphenesin, dehydroacetic acid, diazolidinyl urea, DMDM hydantoin, imidazolidinyl urea, iodopropynyl butylcarbamate, methylchloroisothiazolinone, methylisothiazolinone, methylparaben, and propylparaben.

### F. Optional Additives

In some embodiments, the composition may include additional optional additives to further enhance its properties, typically up to about 10% by weight of the total composition. These may include skin conditioning agents (e.g., niacinamide, panthenol), exfoliants (e.g., finely milled oat powder or other mild abrasives), colorants, or film-forming agents. Further additives may include volatile carriers or performance agents such as terpenes (e.g., d-limonene, pinene) to modify evaporation behavior or enhance cleansing. The extensive list of cosmetic ingredients provided in the record serves as a non-limiting illustration of the types of additional agents that could be incorporated by one skilled in the art without departing from the scope of the invention.

### III. Performance Metrics

The disclosed invention has a demonstrated efficacy for cleaning skin surfaces. The viscosity of the formulation before application to the skin is such that it has the appearance of a thick cream, while after mechanical rubbing the viscosity increases to a high viscosity and loses its cohesion to the skin.

In specific embodiments, the composition is formulated from naturally derived ingredients and biodegrades, providing an environmental utility. To achieve this profile, the formulation may utilize biodegradable film-forming polymers such as tapioca starch or oat starch, natural gums including xanthan gum or konjac gum, and cellulose derivatives like hydroxyethylcellulose. Additionally, the composition may incorporate environmentally benign surfactants such as sodium cocoyl glutamate or decyl glucoside, and plant-derived emollients such as ethyl macadamiate or jojoba esters, ensuring that the residues detached from the skin do not persist in the environment.

### IV. Exemplary Embodiment

The following formulation represents a specific, preferred embodiment of the invention.

### Example 1

A waterless cleansing composition was prepared using the following ingredients at the specified weight percentages:
RO Water: 79.30%
Hydroxyethylcellulose (HEC): 4.00%
Organic Xanthan Gum: 1.00%
Floramac 10 (Ethyl Macadamiate): 8.00%
Tapioca (Arrowroot) Starch: 4.00%
Linalool: 0.20%
Sodium Cocoyl Glutamate: 2.00%
Geogard Ultra (Gluconolactone & Sodium Benzoate): 1.50%

### V. Alternative Embodiments

The scope of the invention is not limited to the specific formulation of Example 1. The following non-limiting examples illustrate alternative embodiments that demonstrate the flexibility of the formulation and provide explicit support for broader protection against simple ingredient substitution.

### Example 2: Alternative Rheology System

A composition is prepared wherein the rheology system of HEC, xanthan gum, and tapioca starch is replaced with a binary blend of other polymers. The formulation comprises approximately 83% water, 3% pullulan, 1.5% konjac gum, 12% ethyl macadamiate, 4% sodium cocoyl glutamate, 2.5% Geogard Ultra, and 0.5% linalool. This demonstrates that the cleansing mechanism can be achieved with different thickening agents.

### Example 3: Alternative Emollient System

A composition is prepared wherein the single emollient, ethyl macadamiate, is replaced with a blend of emollients to achieve a different sensory profile. The formulation comprises approximately 85% water, the rheology system of Example 1, a blend of 7% squalane and 5% jojoba esters, 4% sodium cocoyl glutamate, and 2.5% Geogard Ultra. This shows that the invention is not dependent on a single specific emollient.

### Example 4: Alternative Surfactant System

A composition is prepared wherein the single surfactant, sodium cocoyl glutamate, is replaced with a blend of two different mild surfactants. The formulation comprises approximately 85% water, the rheology and emollient systems of Example 1, and a surfactant system of 3% decyl glucoside and 2% sodium lauroyl sarcosinate, along with 2.5% Geogard Ultra. This demonstrates the interchangeability of the cleansing agents.

### Example 5: Alternative Starch Component

A composition is prepared wherein the tapioca starch is replaced with oat starch, which can function as both a texture enhancer and a bioactive, skin-soothing agent. The formulation comprises approximately 85% water, 8% HEC, 3% xanthan gum, 8% oat starch, 12% ethyl macadamiate, 4% sodium cocoyl glutamate, and 2.5% Geogard Ultra.

### VI. Method of Use

The composition is designed for simple, convenient, and effective waterless cleaning. The method of use comprises the following steps:
1. A small amount of the composition is dispensed onto the surface to be cleaned, for example, the user's hands.
2. The user rubs the composition over the entire surface, similar to washing with conventional soap. This rubbing action serves two purposes: it allows the surfactant to lift dirt and impurities, and it provides the mechanical energy needed to initiate the coagulation of the polymer matrix.
3. As rubbing continues for a short period, typically between 30 seconds and 1.5 minutes, the composition transforms from a gel into a plurality of small, solid, peelable coagulants, aggregates, or particles. These particles encapsulate the impurities removed from the surface.
4. The solid particles lose their cohesion and detach from the surface and fall away, leaving the surface clean, dry to the touch, and with a smooth, conditioned feel.
5. No subsequent rinsing with water or wiping with a towel is necessary to remove any residue, as the product is designed to be self-removing.

What is claimed is:

1. A rinse-free cleansing composition comprising:
(a) an aqueous carrier present in an amount of about 50% to about 90% by weight of the total composition;
(b) a rheology system present in an amount of about 1% to about 12% by weight of the total composition, said rheology system comprising at least one hydroxyalkylcellulose derivative, at least one gum, and at least one film-forming polymer;
(c) an emollient present in an amount of about 1% to about 15% by weight of the total composition;
(d) a mild surfactant present in an amount of about 0.5% to about 6% by weight of the total composition; and
(e) a preservative system;
wherein the composition, when rubbed on a surface, rapidly forms a plurality of solid coagulants that encapsulate impurities and detach from the surface.

2. The composition of claim 1, wherein the hydroxyalkylcellulose derivative is hydroxyethylcellulose.

3. The composition of claim 1, wherein the gum is selected from the group consisting of xanthan gum, guar gum, and konjac gum.

4. The composition of claim 1, wherein the film-forming polymer is selected from the group consisting of tapioca starch, cornstarch, oat starch, polyvinyl alcohol, and polyacrylates.

5. The composition of claim 1, wherein the emollient is ethyl macadamiate.

6. The composition of claim 1, wherein the mild surfactant is an amino acid-based surfactant.

7. The composition of claim 6, wherein the amino acid-based surfactant is sodium cocoyl glutamate.

8. The composition of claim 1, wherein the preservative system comprises gluconolactone and sodium benzoate.

9. The composition of claim 1, wherein:
the aqueous carrier is water present in an amount of about 75% to about 85% by weight;
the hydroxyalkylcellulose derivative is hydroxyethylcellulose present in an amount of about 3% to about 5% by weight;
the gum is xanthan gum present in an amount of about 0.5% to about 1.5% by weight;
the film-forming polymer is tapioca starch present in an amount of about 3% to about 5% by weight;
the emollient is ethyl macadamiate present in an amount of about 5% to about 12% by weight;
the mild surfactant is sodium cocoyl glutamate present in an amount of about 1% to about 4% by weight; and
the preservative system comprises gluconolactone and sodium benzoate and is present in an amount of about 0.3% to about 2.5% by weight.

10. A method for cleaning a surface without rinsing, the method comprising the steps of:
(a) applying a composition to the surface, said composition comprising:
(i) an aqueous carrier;
(ii) a rheology system comprising a hydroxyalkylcellulose derivative, a gum, and a film-forming polymer;
(iii) an emollient; and
(iv) a mild surfactant;
(b) rubbing the composition on the surface for a period of time sufficient to form a plurality of solid, peelable coagulants with increased viscosity that encapsulate impurities from the surface; and
(c) allowing the plurality of solid, peelable coagulants to lose cohesion and detach from the surface, thereby cleaning the surface.

11. The method of claim 10, wherein the period of time is less than two minutes.

12. The method of claim 10, wherein the surface is human skin.

13. The method of claim 10, wherein the composition applied in step (a) comprises:
(i) water present in an amount of about 75% to about 85% by weight;
(ii) hydroxyethylcellulose present in an amount of about 3% to about 5% by weight;
(iii) xanthan gum present in an amount of about 0.5% to about 1.5% by weight;
(iv) tapioca starch as the film-forming polymer present in an amount of about 3% to about 5% by weight;
(v) ethyl macadamiate present in an amount of about 5% to about 12% by weight;
(vi) sodium cocoyl glutamate present in an amount of about 1% to about 4% by weight; and
(vii) a preservative system comprising gluconolactone and sodium benzoate present in an amount of about 0.3% to about 2.5% by weight.

14. A biodegradable, rinse-free cleansing composition formulated from naturally derived ingredients, comprising:
(i) an aqueous carrier;
(ii) a rheology system comprising a hydroxyalkylcellulose derivative, a natural gum, and a plant-derived starch;
(iii) a plant-derived emollient;
(iv) a naturally derived surfactant; and (e) a preservative system; wherein the composition forms a plurality of solid, biodegradable coagulants upon rubbing that encapsulate impurities and detach from the surface.
